(19) United States
(12) Patent Application Publication
(10) Pub. No.: US 2013/0066236 A1
(43) Pub. Date: Mar. 14, 2013

(54) MOUTHGUARD APPARATUS AND RELATED METHOD

(71) Applicants: Mark H. Herman, Minneapolis, MN (US); William Cook, Lakeville, MN (US); Lawrence Calcano, Greenwich, CT (US)

(72) Inventors: Mark H. Herman, Minneapolis, MN (US); William Cook, Lakeville, MN (US); Lawrence Calcano, Greenwich, CT (US)

(73) Assignee: BITE TECH, INC., Norwalk, CT (US)

(21) Appl. No.: 13/701,483

(22) PCT Filed: May 31, 2011
(86) PCT No.: PCT/US11/38646

§ 371 Date: Nov. 30, 2012

Related U.S. Application Data
(60) Provisional application No. 61/350,243, filed on Jun. 1, 2010.
(60) Provisional application No. 61/350,231, filed on Jun. 1, 2010.

(51) Int. Cl. A61C 5/14; A61B 5/11; A61B 5/00

(52) U.S. Cl. 600/595; 128/861; 600/300; 128/862

(57) ABSTRACT

A mouthguard apparatus is disclosed herein. In various aspects, the mouthguard apparatus includes an electronics package adapted to attach removably to a dental appliance. The dental appliance may be configured to be secured within the mouth of a user. At least portions of the electronics package adapted to extend external to a mouth of the user when the electronics package is attached to the dental appliance and the dental appliance is secured within the mouth of the user, in various aspects. Related methods are disclosed herein. This Abstract is presented to meet requirements of 37 C.F.R. §1.72(b) only. This Abstract is not intended to identify key elements of the apparatus and methods disclosed herein or to delineate the scope thereof.

### CROSS-REFERENCE TO RELATED APPLICATIONS

This application claims the priority and benefit of U.S. Provisional Patent Application 61/350,231 filed 1 Jun. 2010 and entitled “INTERCHANGABLE MOUTHGUARD COMPONENT SYSTEM”, the priority and benefit of U.S. Provisional Patent Application 61/350,243 filed 1 Jun. 2010 and entitled “MOUTHGUARD FITMENT TOOL SYSTEM,” which are incorporated herein by reference in their entirety.

### BACKGROUND OF THE INVENTION

1. Field

The present disclosure relates to oral apparatus, and, more particularly, to oral apparatus and associated methods for monitoring the user of the oral apparatus.

2. Background

Various dental appliances including mouthguards may be removably received within the mouth of the user in order to confer protection upon the user while the user is engaged in various sporting or other physical activities. The dental appliances may offer various therapeutic or other benefits to the user apart from protection, in various aspects. In addition, the dental appliances may include various electronic components that may be used to monitor the condition of the user. Other electronic components may be disposed about the user that may be used to monitor the condition of the user.

For example, it may be desirable to measure impacts upon the user such as impacts that result from engagement in a sport. While various sensors that measure impacts may be disposed about a helmet or garment of the user, a direct connection of the sensor to the user's head may be desirable for measurement of impacts upon the user's head including the neck.

The electronic components included in the dental appliance may receive power from a power source that may include a battery. It may be desirable, for example, to position the battery portion of the power source externally to the user while the user wears the dental appliance, as the battery may be toxic or otherwise injurious to the user.

Various forces may be applied to the dental appliance while the dental appliance is in use that may lead to failure of the dental appliance. For example, the dental appliance may be subjected to mechanical forces applied to the user as a consequence of the user's participation in sport or other activity or merely as a result of routine life events. The user may bite upon the dental appliance. The user may remove the dental appliance from its secured position in the mouth while retaining the dental appliance generally in the mouth, and then chew the dental appliance, manipulate the dental appliance with the lips, and so forth. Such actions and their attendant forces, as well as other forces and actions to which the dental appliance may be subjected, may result in failure of the dental appliance.

When electronic components are included in the dental appliance to monitor the condition of the user, these electronic components are rendered inoperative for their intended purpose when the dental appliance fails. Similarly, the electronic components may fail, which renders the dental appliance with the electronic components embedded therein inoperative for its intended purpose. Accordingly, it may be desirable to make the electronic components, at least in part, replaceable with respect to the dental appliance, and to make the dental appliance replaceable with respect to at least a portion of the electronic components. When the dental appliance fails, a new dental appliance may be secured to at least a portion of the electronic components that remain functional, or, when the electronic components fail, the electronic components may be removed from the dental appliance and replaced in the dental appliance with functional electronic components.

Accordingly, there may be a need for improved mouthguard apparatus and related methods that may satisfy at least some of the above noted needs, or that may overcome at least some of the above noted disadvantages.

### BRIEF SUMMARY OF THE INVENTION

These and other needs and disadvantages may be overcome by the mouthguard apparatus, component system and related methods of operation disclosed herein. Additional improvements and advantages may be recognized by those of ordinary skill in the art upon study of the present disclosure.

A mouthguard apparatus is disclosed herein. In various aspects, the mouthguard apparatus includes an electronics package adapted to attach removably to a dental appliance. The dental appliance may be configured for securement within the mouth of a user and may include at least one bite surface. At least portions of the electronics package adapted to extend external to a mouth of the user when the electronics package is attached to the dental appliance and the dental appliance is secured within the mouth of the user, in various aspects. Related methods are disclosed herein.

This summary is presented to provide a basic understanding of some aspects of the apparatus and methods disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1 illustrates by an exploded perspective view an exemplary implementation of a mouthguard apparatus;

FIG. 2 illustrates by bottom view the exemplary implementation of the mouthguard apparatus of FIG. 1;

FIG. 3A illustrates by perspective view portions of the exemplary implementation of the mouthguard apparatus of FIG. 1 including a male attachment and corresponding female attachment;

FIG. 3B illustrates by top cut-away view portions of the exemplary implementation of the mouthguard apparatus of FIG. 1 including the male attachment received within the female attachment;

FIG. 3C illustrates by side cut-away view portions of the exemplary implementation of the mouthguard apparatus of FIG. 1 including the electronics package attached to the dental appliance;

FIG. 3D illustrates by side view the electronics package secured to the dental appliance in the exemplary implementation of the mouthguard apparatus of FIG. 1;

FIG. 4A illustrates by top view an exemplary implementation of the electronics package of the mouthguard apparatus of FIG. 1 secured to a strap;

FIG. 4B illustrates by side cut-away view a detail of an exemplary implementation of the attachment of the electronics package to the strap;

FIG. 5 illustrates by top view another exemplary implementation of an electronics package;

FIG. 6 illustrates by schematic block diagram an exemplary implementation of a mouthguard apparatus including an electronics package in communication with electronic circuitry disposed within a dental appliance;

FIG. 7 illustrates by perspective view the exemplary implementation of the mouthguard apparatus of FIG. 6 in use by a user;

FIG. 8 illustrates by perspective view an exemplary plug for use in a mouthguard apparatus;

FIG. 9 illustrates by perspective view an exemplary handle for manipulation of a mouthguard apparatus;

FIG. 10 illustrates by schematic block diagram an exemplary implementation of a mouthguard apparatus including an electronics package removably securable to a dental appliance;

FIG. 11 illustrates by schematic block diagram an exemplary implementation of a mouthguard apparatus including an electronics package in communication with electronic circuitry integrated with a dental appliance;

FIG. 12A illustrates by top view a portion of an exemplary implementation of a mouthguard apparatus including an electronics package secured to a strap;

FIG. 12B illustrates by top view a portion of another exemplary implementation of a mouthguard apparatus including a strap with electronics packaged disposed at both ends thereof; and

FIG. 13 illustrates by process flow chart an exemplary method of use of a mouthguard apparatus.

The Figures are exemplary only, and the exemplary implementations illustrated therein are selected to facilitate explanation. The number, position, relationship and dimensions of the elements shown in the Figures to form the various exemplary implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the exemplary implementations shown in the drawings and are utilized to facilitate description thereof.

### DETAILED DESCRIPTION OF THE INVENTION

A mouthguard apparatus and associated methods are disclosed herein for sensing the user status of a user including impacts upon the user. The user status of the user may include, for example, a physiological state of the user, a spatial location of the user, orientation of the user, sounds or movements emanating from the user, or an impact such as a blow to the user's head, neck, other parts of the user, or combinations thereof, in various aspects.

The mouthguard apparatus may include a dental appliance and an electronics package. The dental appliance disclosed herein is generally receivable within the mouth of a user, and is removably securable to at least a portion of the teeth of the user, in various aspects. The dental appliance may include at least one bite surface that may be positioned about the user's teeth. In various aspects, the dental appliance is configured as a mouth guard for the protection of the teeth, tongue, lips, mandible, or other anatomical features of the mouth, jaw, or face. The dental appliance may support various anatomical features of the mouth, jaw, or face such as, for example, the temporomandibular joint, in various aspects. The dental appliance, in various aspects, may perform other functions, such as, for example, the reduction of snoring, as would be recognized by those of ordinary skill in the art upon study of this disclosure.

An electronics package may be removably attached to the dental appliance. With the electronics package attached to the dental appliance and with the dental appliance positioned within the user's mouth, at least portions of the electronics package may be positioned external to the mouth, in various aspects.

The electronics package may include at least a portion of a power source that provides electrical power to the electronics package, and at least portions of the power source, such as a battery, may be positioned external to the mouth of the user when the dental appliance is engaged with the mouth. The electronics package may include at least a portion of a transceiver, and at least portions of the transceiver may be positioned external to the mouth of the user when the dental appliance is engaged with the mouth. The electronics package may be adapted to detect the user status of the user. One or more sensors may be included in the electronics package to detect the user status, in various aspects.

In some aspects, the electronics package may be operable to determine independently the user status of the user. That is, the sensor, power source, transceiver, and other electronic components are contained entirely in the electronics package. In other aspects, the dental appliance may include electronic circuitry adapted to communicate electrically with the electronics package, and the electronic circuitry may cooperate with the electronics package to determine user status of the user. The electronic circuitry may cooperate with the electronics package to transmit via a transceiver signals indicative of the user status of the user, in various aspects. The transceiver may be included, at least in part, in the electronics package, the electronic circuitry, or both the electronics package and the electronic circuitry, in various aspects. The electronic circuitry may cooperate with the electronics package to receive power from the power source, in various aspects. The electronic circuitry may cooperate with the electronics package to regulate the delivery of power from the power source to, for example, the sensor, the transceiver, or other elements of the electronics package, the electronic circuitry, or both, in various aspects.

The electronic circuitry may include the sensor that, for example, detects the user status of the user and generates sensor data indicative of the user status. For example, the sensor may detect physiologic indicators that are indicative of user status such as, for example, the user's body temperature, pulse rate, cardiac rhythm, breathing rate, CO₂ in the user's breath, electrical activity such as neurological activity, or various other physiologic indicators or combinations of physiologic indicators that may be present in the user's breath, saliva, electrical activity, tongue, teeth, pallet, gums, lips, blood, or otherwise detectable about the user's mouth or head. The sensor may generate a data signal indicative of the physiologic indicator. The sensor may be embedded in the one or more material layers, or may be secured about the surface of the dental appliance such that portions of the sensor are exposed, or both in various aspects. The sensor may be positioned about the dental appliance to interact with the user at a specified location that may be conducive to detection of, for example, a particular physiologic indicator such as heart rate.

The sensor may be affixed to the mouthguard, to the electronics package that is affixed to the mouthguard, or both, and the mouthguard is secured to the upper mandible or the lower mandible, in various aspects, so that the sensor is in generally mechanical communication with the user. Because the sensor is in generally mechanical communication with the user, user status including motions of the user, the position of the user, and impacts upon the user are communicated to the sensor through the mechanical communication. The user status may be communicated to the sensor through the rigid mechanical connection without substantial damping, phase lag, or other disconnect between the sensor and the user status.

For example, sensor may detect user status in the form of the position of the user with respect to a reference x-y-z coordinate system such as a global position system (GPS). The sensor may, for example, detect user status in the form of the user's orientation with the vertical or horizontal, and such orientation may be relatable to a position, orientation, or spatial location of the user, or combination thereof.

As yet another example, the sensor may detect user status in the form of an impact upon the user, such as a blow to the user's head, in various aspects. The sensor may detect the magnitude of the impact, or the sensor may detect the user's response to the impact, or both, in various aspects. For example, the sensor may detect the motion of the user's head, including the neck and upper extremities, when this motion is induced by an impact to the user's head. The impact to the user's head may result from the user's participation in a sport such as football, hockey, lacrosse, rugby, and so forth. The user status may, for example, include the magnitude of the impact, the user's response to the impact, or both may enable determination of the user status of the user. Impacts and various blows delivered to the user or by the user may be communicated to the sensor that is in substantially rigid mechanical communication with the user.

The dental appliance may be constructed of a plurality of material layers bonded or otherwise secured to one another in various ways. For example, the material layers may include a material layer formed as a base, and may include a material layer formed as an occlusal pad. The dental appliance may be constructed, at least in part, of the base bonded to the occlusal pad, and the base with the occlusal pad bonded thereto forms at least a portion of the dental appliance. In various aspects, the base may support the occlusal pad, may interact with other elements of the dental appliance, and may confer various mechanical properties upon the dental appliance. In various aspects, the occlusal pad may engage the user's teeth to secure the dental appliance thereto, and the occlusal pad may be custom fitted to engage the user's teeth.

The base is composed of one or more base materials. In various aspects, the base material may include a mixture of styrene block copolymer and ethylene vinyl acetate (EVA). An exemplary styrene block copolymer is available as DYNAFLEX® part number G2782 from GLS Corporation, Thermoplastic Elastomers Division, 833 Ridgeview Dr., McHenry, Ill. 60050. EVA is available from a number of sources, such as the ELVAX® resins from Dupont Packaging and Industrial Polymers, 1007 Market Street, Wilmington, Del. 19898.

In various aspects, the base material may include a mixture of a styrene block copolymer and a polyolefin elastomer. The polyolefin elastomer may be a copolymer of ethylene and octene-1. An exemplary copolymer is available as ENGAGE® from Dupont Canada, Inc., P.O. Box 2200, Streetsville, Mississauga, Ontario L5M 2H3.

The base material may include, in various aspects, a mixture of a thermoplastic rubber, which includes thermoplastic elastomer and thermoplastic urethane, with a polyolefin elastomer. Exemplary thermoplastic rubbers are Santoprene® thermoplastic elastomer from Advanced Elastomer Systems, L. P., 388 South Main Street, Akron, Ohio 44311 and Kraton® thermoplastic elastomer from the Shell Oil Company, Houston, Tex. Kraton® includes a styrene-ethylenelbutylenes-styrene block copolymer. In various aspects, the base material may include polypropylene part number AP6112-HS from Huntsman Corporation, Chesapeake, Va. 23320. In various aspects, the base material of base 20 may include HD-6706 ESCORENE® Injection Molding Resin [a high density polyethylene] from ExxonMobil Chemical Company, P.O. Box 3272, Houston, Tex. In some aspects, the base material of the base has a Shore A hardness of about 82.

The occlusal pad is composed of one or more occlusal pad materials. The occlusal pad material may be transformable between a pliable state and a non-pliable state. In the pliable state, the occlusal pad material of the occlusal pad may be shaped to conform to the teeth of the user. In the non-pliable state, the occlusal pad material of the occlusal pad generally retains its conformance to the teeth of the user as shaped when in the pliable state. In various aspects, the occlusal pad may be transformed between the pliable state and the non-pliable state by heating and cooling, respectively. For example, heating the occlusal pad material in warm water may allow the occlusal pad to be fitted to the user's teeth and, after having been fitted, the occlusal pad material may be cooled to the non-pliant state thereby capturing the fit of the user's teeth in the occlusal pad material. The occlusal pad material is transformed from the non-pliable state to the pliable state at a temperature tolerable by the user upon placement of the occlusal pad material in the pliable state within the user's mouth, in various aspects. The occlusal pad material may transform between the non-pliant and pliant state at a temperature greater than human body temperature but less than about 100° C., in various implementations.

In various aspects, the occlusal pad material of the occlusal pad includes a mixture of polycaprolactone. An exemplary polycaprolactone is Capra 6500 polycaprolactone from Perstorp, UK Limited, Warrington, Cheshire UK. In various aspects, the occlusal pad material of occlusal pad 40 includes a mixture of polycaprolactone and ethylene vinyl acetate (EVA) such as ELVAX®. In various aspects, the occlusal pad material of occlusal pad 40 includes ethylene vinyl acetate (EVA) alone, such as ELVAX®. In various aspects, the occlusal pad material of occlusal pad 40 includes a mixture of polycaprolactone and a polyolefin elastomer, and the polyolefin elastomer may be a copolymer of ethylene and octene-1. An exemplary copolymer is available as ENGAGE® from Dupont Canada, Inc., P.O. Box 2200, Streetsville, Mississauga, Ontario L5M 2H3.

The electronic circuitry may be attached at least in part to the surface of the dental appliance, embedded at least in part within at least one material layer of the dental appliance, or both attached to the surface of the dental appliance and embedded within at least one material layer of the dental appliance. For example, the electronic circuitry may be embedded in the base, and portions of the electronic circuitry distributed about a surface of the base. In aspects having multiple material layers, the electronic circuitry may be embedded in a single material layer of the material layers, or, alternatively, the electronic circuitry may be distributed among the material layers in various ways. For example, in aspects of the dental appliance having a base and an occlusal pad, at least portions of the electronic circuitry may be embedded in the occlusal pad, and the remaining portions, if any, of the electronic circuitry may be embedded in the base. In aspects of the dental appliance having material layer(s) in addition to the base and the occlusal pad or in aspects of the dental appliance having material layer(s) other than the base and the occlusal pad, the electronic circuitry may be embedded, at least in part, within those additional material layers or other material layer(s).

In various aspects, the electronics package may be removed from the dental appliance and an alternate electronics package attached to the dental appliance to confer different capabilities upon the mouthguard apparatus. In various aspects, when the electronics package fails, the electronics package may be removed from the dental appliance and a functional electronics package attached to the dental appliance in replacement of the failed electronics package. Conversely, if the dental appliance fails, including structural failure or failure of the electronics circuitry, the dental appliance may be replaced with an alternate dental appliance and the electronics package attached to the alternate dental appliance. Elements of the mouthguard apparatus may be allocated between the electronics package and the dental appliance that may enhance the disposability of either the electronics package or of the dental appliance, in various aspects.

In various aspects, kits may be provided may include a plurality of electronics packages that may be attachable to a particular dental appliance, each electronics package of the plurality of electronics packages having particular attributes. The kits may include an electronics package attachable to a plurality of dental appliances, each dental appliance of the plurality of dental appliances having a particular attribute. The kits may include a plurality of electronics packages, a plurality of dental appliances that may be interchanged to configure various combinations of electronics packages and dental appliances having a variety of attributes.

In various aspects, the electronics package may be removed from the dental appliance and a plug inserted into the dental appliance in replacement of the electronics package. The plug may be electronically non-functional.

The electronics package, the plug, or both the electronics package and the plug may include a display surface, and an identifying mark, slogan, name, logo, combinations thereof, or so forth, may be displayed upon the display surface, in various aspects. The electronics package, the plug, or both the electronics package and the plug may be securable to a tether. The tether may, in turn, be securable to, for example, various portions of the user's clothing or equipment such as the user's helmet or faceguard, in various aspects, to secure thereto the electronics package and dental appliance attached to the electronics package.

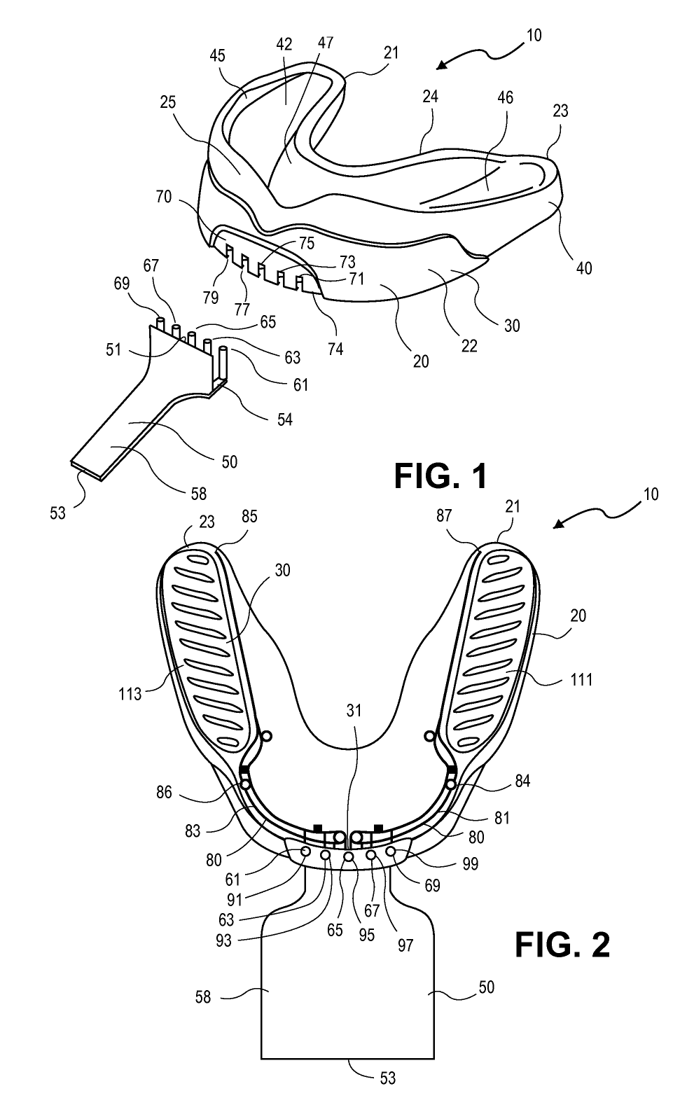

With reference to the exemplary implementations of the Figures, FIG. 1 illustrates an implementation of the mouthguard apparatus 10. As illustrated in FIG. 1, the mouthguard apparatus 10 includes dental appliance 20 and electronics package 50, with electronics package 50 illustrated as removed from the dental appliance 20. The dental appliance 20, as illustrated, is formed in a generally U-shaped structure passing between end 21 and end 23. In this implementation, the dental appliance includes base 30 and occlusal pad 40 with occlusal pad 40 bound to the base 30. Other implementations (not shown) may include only one structure such as a base and the structure may be formed of a single material or single combination of materials. For example, the dental appliance may be formed as a single structure of a ruberous material. Still other implementations of the dental appliance (not shown) may include multiple structures in addition to the base structure bound to occlusal pad structure of the implementation illustrated in FIG. 1.

The occlusal pad 40, in this implementation, defines channel 42 generally bounded by opposing walls 45, 47 with bottom 46, all formed of occlusal pad material. Channel 42 may receive one or more teeth therein in order to removably secure the dental appliance 20 within the mouth to the one or more teeth of the user. With the teeth engaged in channel 42, the dental appliance 20 is generally frictionally secured to the teeth. In this implementation, channel 42 of dental appliance 20 is oriented to engage teeth of the upper jaw to secure dental appliance 20 to the teeth of the upper jaw. With channel 42 of implementation of dental appliance 20 secured to the teeth of the upper jaw, ends 21, 23 of dental appliance 20 are located posterially within the mouth, for example, about the molars, so that the teeth of the user anterior of the molars engaged with ends 21, 23 are received within channel 42. Side 22 of dental appliance 20 is oriented in the buccal-labial direction, side 24 of dental appliance 20 is oriented in the lingual direction, and middle portion 25, which is medial between ends 21, 23, is oriented toward the user's lips (i.e. labially), in this implementation. In other implementations (not shown), the channel, such as channel 42 could be oriented to engage the teeth of the lower jaw to secure the dental appliance to the teeth of the lower jaw.

In implementations with occlusal pad 40 formed of occlusal pad material that is transformable between a pliable state and a non-pliable state, channel 42 of occlusal pad 40 of dental appliance 20 may be custom fit to a particular user. For example, the dental appliance 20 may be heated by placement in hot water to soften the occlusal pad material of the occlusal pad 40 into a pliable state. With occlusal pad material of occlusal pad 40 in the pliable state, the user may position dental appliance 20 in the mouth such that the user's teeth are placed within channel 42 and the dental appliance 20 is otherwise properly positioned in the user's mouth. With the dental appliance so properly positioned, the user may then bite to force the occlusal pad 40 including channel 42 into fitment to the user's teeth. When the user bites, the occlusal pad material, which is in the pliable state, deforms until the occlusal pad 40 including channel 42 assumes a shape that conforms to the user's teeth—i.e. the shape of the user's teeth is impressed into the occlusal pad 40 so that channel 42 conforms to the user's teeth. The temperature at which the occlusal pad material of the occlusal pad 40 becomes pliant is, for example, generally tolerable by the user when the user engages in this fitting process. Tolerable to the user, in various implementations, means unobjectionable to the user and non-injurious to the user. After the occlusal pad 40 has conformed to the teeth, the user may cool the occlusal pad material of the occlusal pad 40 by, for example, taking cool water into the mouth. Upon being cooled, the occlusal pad material is transformed into the non-pliant state in which the occlusal pad material recovers sufficient rigidity to retain its shape, which now conforms to the user's teeth as imprinted upon the occlusal pad by biting thereupon. The dental appliance is now fitted to the user's mouth.

The implementation of the mouthguard apparatus 10 illustrated in FIG. 1 includes electronics package 50. Electronics package 50 may be secured about recess 70 in base 30 proximate middle portion 25 of dental appliance 20, in this implementation. As illustrated in FIG. 1, end 51 of electronics package 50 includes male fittings 61, 63, 65, 67, 69 that may be removably received within female fittings 71, 73, 75, 77, 79, respectively to removably secure electronics package to dental appliance 20. With male fittings 61, 63, 65, 67, 69 received within female fittings 71, 73, 75, 77, 79, portions of the electronics package 50 proximate end 51 may be formed to fit within recess 70 and otherwise contoured such that a generally smooth surface transition is formed between base 30 of dental appliance 20 and electronics package 50. In other implementations, female fittings may be disposed about electronics package 50 to secure removably electronics package 50 to corresponding male fittings on dental appliance 20. In yet other implementations, combinations of male fittings and female fittings may be disposed about electronics package 50 to secure removably electronics package 50 corresponding fittings on the dental appliance 20. In other implementations, other fittings may be positioned about the electronics package 50 and corresponding fittings positioned about the dental appliance 20 to allow the electronics package 50 to be removably secured to the dental appliance 20.

With the electronics package 50 secured to the dental appliance 20 and dental appliance 20 positioned within the mouth of the user, the electronics package 50 is secured to the jaw of the user. The electronics package may extent forth from the user's mouth such that at least portions of electronics package 50 proximate end 53 lie outside the user's mouth when electronics package 50 is secured to the dental appliance 20 and dental appliance 20 positioned within the mouth of the user. Accordingly, when so positioned, portions of the electronics package 50 may lie between the user's lips, and the user's lips may be generally biased about portions of the electronics package 50. The electronics package 50 may be conformed in various ways to facilitate placement between the lips, such as by being curved, being of a width or thickness that accommodates placement between the lips, being surfaced with various textures, and so forth, in various implementations. Various logos or other displays may be formed about the surface of the electronics package 50, in various implementations, and the logos may be, at least in part, positioned external to the user's mouth.

The electronics package 50 may include various electronic components such as, for example, a transceiver, a video camera, audio device, or a power source such as a battery. In various implementations, the electronics package 50 is formed such that at least portions of the electronic components within electronics package 50 are positioned external to the user when the mouthguard apparatus 10 is engaged with the user's mouth.

FIG. 2 illustrates mouthguard apparatus 10 with electronics package 50 secured to dental appliance 20. As illustrated in FIG. 2, the traction pads 111, 113 are disposed about base 30 of dental appliance 20 to contact lower posterior teeth of the user when dental appliance 20 is positioned in the mouth. Traction pads 111, 113 are formed to prevent slippage between the posterior teeth and the dental appliance 20. Dental appliance 20 includes electronic circuitry 80 that branches into circuit 83 that extends generally toward end 23 of dental appliance 20, and branches into circuit 81 that extends generally toward end 21 of dental appliance 20. The electronic circuitry 80 includes sensor 84 that communicates with circuit 81 and sensor 86 that communicates with circuit 83, in this implementation. The electronic circuitry 80 may include fewer or additional branches, such as branches 81, 83. The electronic circuitry 80 may include any number of sensors, such as sensors 81, 83, and the sensor(s) may be located in various locations about the electronic circuitry. In some implementations, the electronic circuitry 80 may include no sensors. Various electrical devices may be included in the electronic circuitry 80, in various implementations.

As illustrated in FIG. 2, electronics package 50 is secured to dental appliance 20. Contacts 91, 93, 95, 97, 99 are formed about male fittings 61, 63, 65, 67, 69, respectively, to enable electrical communication between electronics circuitry 80, and electronic components within electronics package 50 in this implementation. Contacts 91, 93, 95, 97, 99 may be formed in various ways about end 51 of electronics package 50 and face 70 of dental appliance 20, in various implementations, for electrical communication between electronics package 50 and electronics circuitry 80.

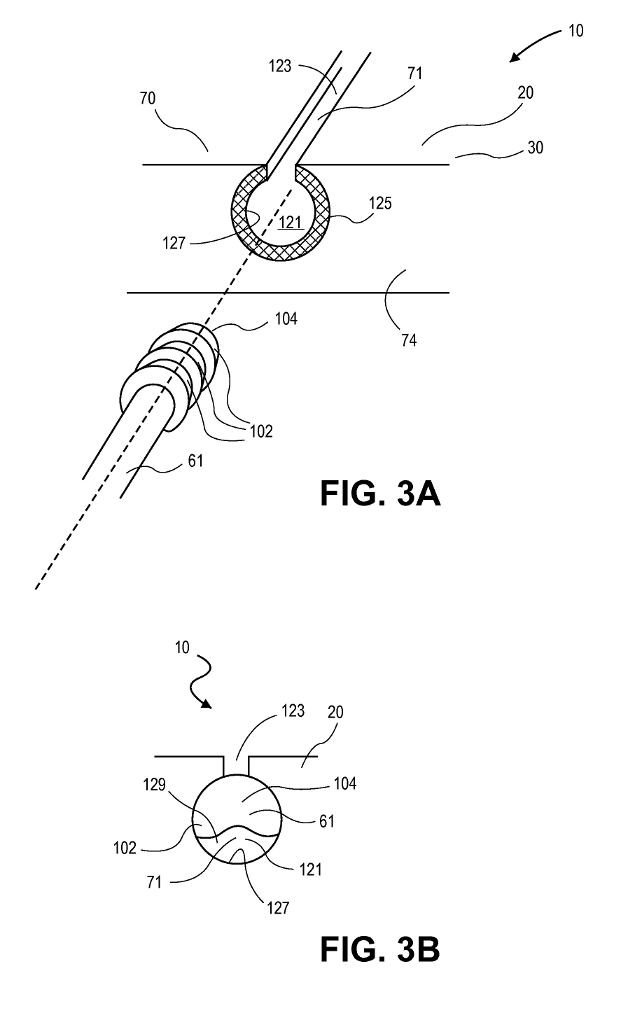

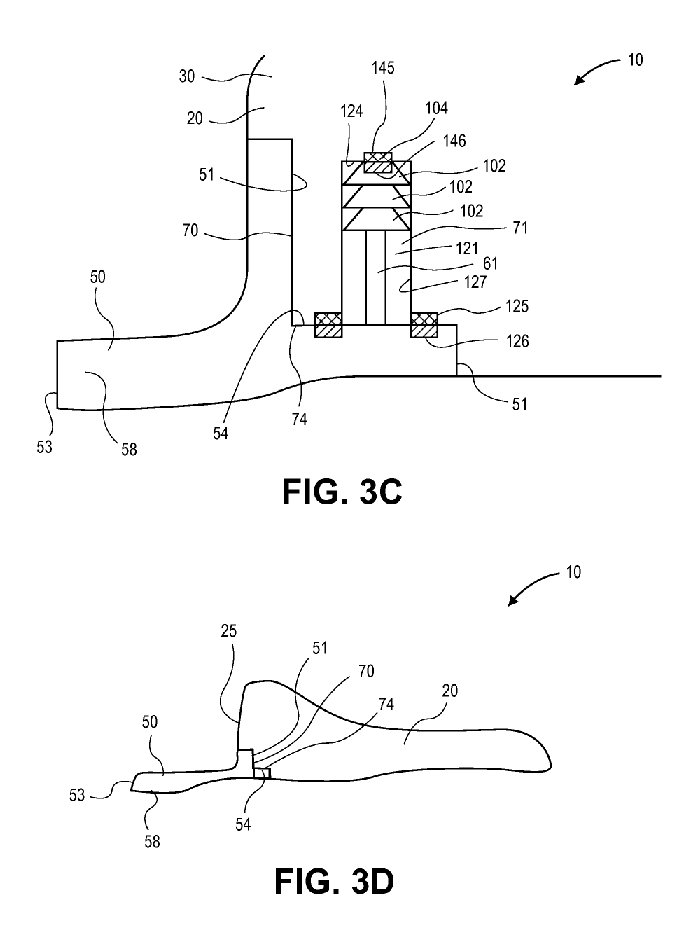

FIG. 3A illustrates an implementation of male fitting 61 and corresponding female fitting 71. Male fittings 63, 65, 67, 69 and corresponding female fittings 73, 75, 77, 79 of mouthguard apparatus 10 may be formed in a similar manner as male fitting 61 and female fitting 71, respectively, as illustrated in FIG. 3A. Male fitting 61 includes ridges 102 that extend at least partially circumferentially around male fitting 61 near end 104 of male fitting 61. In other implementations, ridges, such as ridges 102 may be variously positioned about male fitting 61 in various relationships with end 104. Ridges 102 may surround about 40% to about 60% of the circumference of the male fitting 61, in various implementations. In various implementations, ridges 102 may surround about 20% to about 80% of the circumference of the male fitting 61. In various implementations, ridges 102 may surround about 30% to about 70% of the circumference of the male fitting 61.

As illustrated in FIG. 3A, female fitting 71 includes passage 121 of base 30 of dental appliance 20 and slot 123. Contact 125 surrounds passage 121, as illustrated, to provide electrical communication with the electronics package 50 with the male fitting 61 fully received in female fitting 71 (see FIG. 3C). Slot 123 extends generally along the length of passage 121 to vent passage 121 to the external environment. As illustrated, male fitting 61 may be received within passage 121 of female fitting 71 such that ridges 102 are embraced by the sidewall 127 of passage 121 to secure male fitting 61 in female fitting 71 thereby securing electronics package 50 to dental appliance 20. Partial circumferential extension of ridges 102 around male fitting 61 may allow venting of air, which prevents buildup of pressure or development of vacuum within passage 121 as the male fitting 61 is inserted into female fitting 71 or as male fitting 61 is withdrawn from female fitting 71, respectively. This venting of air may facilitate attachment of the electronics package 50 to the dental appliance 20, removal of the electronics package 50 from the dental appliance 20, or both.

FIG. 3B illustrates a detail of ridge 102 proximate end 104 of male fitting 61 received within passage 121 of female fitting. Passage 129, as illustrated, is formed between the boundary of ridge 102 and sidewall 127 of passage 121. Air may pass through passage 129 as the as the male fitting 61 is inserted into female fitting 71 or as male fitting 61 is withdrawn from female fitting 71. As illustrated, air may pass through slot 123 as the as the male fitting 61 is inserted into female fitting 71 or as male fitting 61 is withdrawn from female fitting 71. As illustrated, male fitting 61 is generally cylindrical with generally cylindrical ridges 102 that pass at least partially circumferentially around cylindrical male fitting 61. Female fitting 71 is formed as a cylindrical passage 121 in correspondence to male fitting 61. In other implementations, the male fitting, such as male fitting 61, may have other shapes such as a rectangular post, triangular post, and so forth, the ridge(s), such as ridges 102, may be shaped accordingly, and the female fitting, such as female fitting 71, may be correspondingly shaped to receive the male fitting. Other attachment mechanisms may be employed to attach removably the electronics package 50 to the dental appliance 20 in other implementations.

FIG. 3C illustrates male fitting 61 received within female fitting 71 such that electronics package 50 is secured to dental appliance 20 with faces 51, 54 of electronics package 50 biased against faces 71, 74, respectively, of dental appliance 20. End 104 of male fitting 61 is biased against end 124 of passage 121 of female fitting 71, as illustrated, and portions of ridges 102 are biased against sidewall 127 of passage 121 to frictionally secure male fitting 61 within passage 121 of female fitting 71.

Although not included in the illustration, electronic circuitry 80 communicates with contact 125 and electronic components of electronics package 50 communicate with contact 126. In this implementation, with male fitting 61 inserted as illustrated within female fitting 71, contact 125 on dental appliance 20 is biased against contact 126 on electronics package 50 so that electronic circuitry 80 of dental appliance 20 and electronics package 50 are in electrical communication with one another, as illustrated. Contact 146, which is located at end 104 of male fitting 61, is biased against contact 145 on dental appliance 20 so that dental appliance 20 and electronics package 50 are in electrical communication with one another, as illustrated. In other implementations, contact(s) may be variously located about male fitting 61 and corresponding contact(s) located about passage 121 of female fitting 71. In other implementations, various clips, contact points, and so forth may be positioned, for example, about face 70 of dental appliance 20 or otherwise located about dental appliance 20 with corresponding contacts on electronics package 50 so that the electronics package 50 may electrically communicate with electronic circuitry 80 within dental appliance 20 when the electronics package 50 is attached to dental appliance 20.

FIG. 3D illustrates electronics package 50 secured to dental appliance 20. As illustrated, electronics package 50 is secured to middle portion 25 of dental appliance 20 with faces 51, 54 of electronics package 50 biased against faces 71, 74, respectively, of dental appliance 20. The surfaces of electronics package 50 and dental appliance 20 are formed to make a generally smooth transition between the electronics package 50 and the dental appliance 20. The electronics package 50, as illustrated, is secured to dental appliance 20 and extends past the lips of the user such that portions of the electronics package 50 including end 53 form an external portion 58 of electronics package 50. External portion 58 is positioned external to the mouth when the dental appliance is properly positioned within the user's mouth, in this implementation.

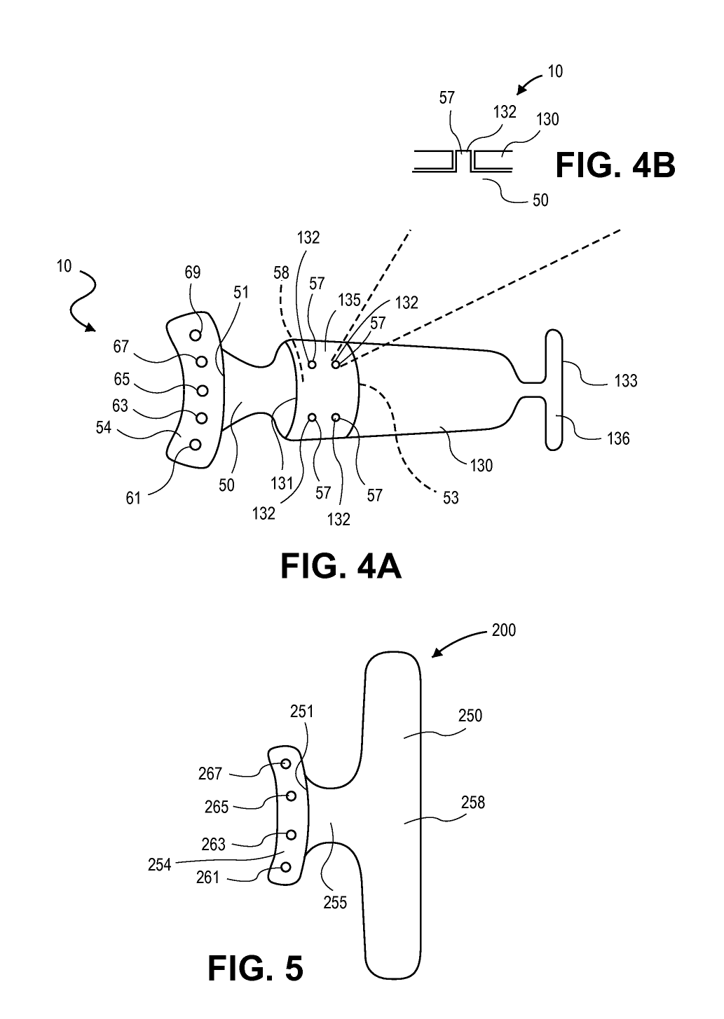

FIG. 4A illustrates electronics package 50 secured to strap 130. As illustrated, strap 130 includes sleeve portion 135 proximate end 131. At least portions of electronics package 50 may be received securely within sleeve portion 135 of strap 131. End 133 of strap 130, which is opposite of end 131 of strap 130, is formed as attachment 136 that may be secured to a helmet, including other piece of equipment or clothing, to secure the strap thereto, thereby securing electronics package 50 and dental appliance 20, if attached to electronics package 50, to the helmet.

FIG. 4B illustrates a detail of the attachment of electronics package 50 to strap 130. As illustrated, with portions of electronics package 50 received within sleeve portion 135 of strap 130, protrusions 57 located about the surface of electronics package 50 are received in corresponding holes 132 located about sleeve portion 135 to secure the electronics package 50 to strap 130.

FIG. 5 illustrates another implementation of electronics package 250 that forms a portion of mouthguard apparatus 200. As illustrated, male fittings 261, 263, 265, 267 are received within corresponding female fittings (not shown) on a corresponding dental appliance (not shown) to secure removably electronics package 250 to the dental appliance. With the electronics package 250 so secured to the dental appliance, faces 251, 254 align with corresponding faces (not shown) on the dental appliance. Electronics package 250, as illustrated, is formed with a narrow section 255 that passes between the lips when the dental appliance with electronics package 250 secured thereto is positioned in the mouth. External portion 258 of electronics package 250 then lies outside the mouth, and external portion 258 is generally aligned parallel to the lips, in this implementation. In other implementations, the electronics package may assume various shapes, and, the electronics package may be aligned with the user's head, face, or jaw in various ways.

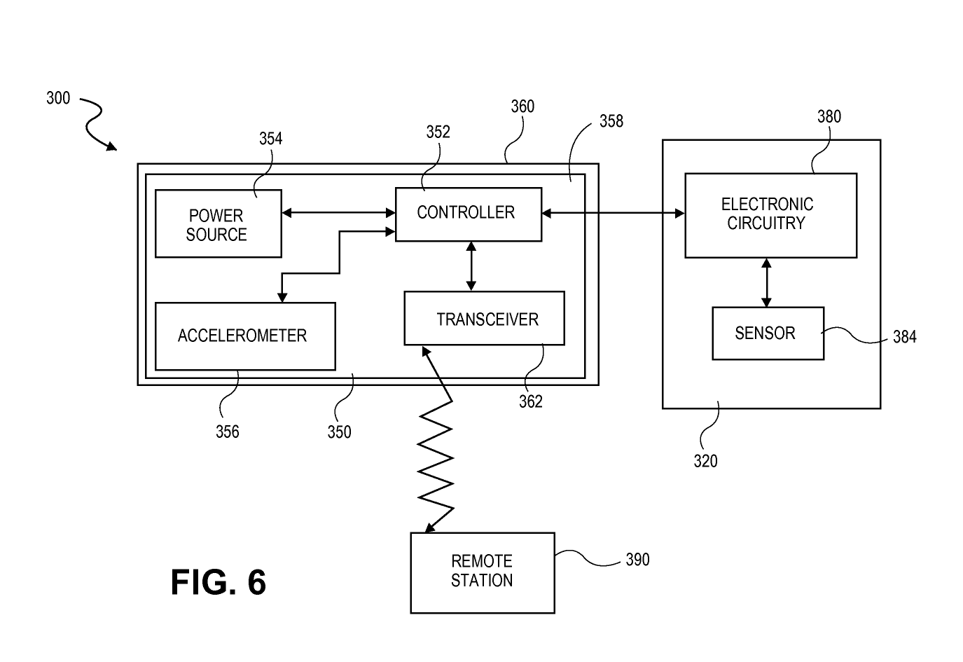

FIG. 6 illustrates an implementation of mouthguard apparatus 300 that includes electronics package 350 in communication with electronic circuitry 380. Sensor 384, as illustrated, is in communication with electronic circuitry 380, and electronic circuitry 380 is included in dental appliance 320. The electronic components 360 within electronics package 350 include controller 352, power source 354, accelerometer 356, and transceiver 362 in this illustrative implementation, but may include other combinations of electronic components in other implementations. Other implementations may include or exclude the sensor 384, the accelerometer 356, or other electronic components, and the sensor 384, the accelerometer 386, or other electronic components may be apportioned between the dental appliance, such as dental appliance 320, and the electronics package, such as electronics package 350, in various ways in various other implementations.

Electronics package 350 may be formed similarly to electronics packages 50, 250, and dental appliance 320 may be formed similarly to dental appliance 20, in various implementations. Electronics package 350 includes an external portion 358. External portion 358 of electronics package 350 may be generally positioned externally to the mouth when electronics package 350 is secured to dental appliance 320 and dental appliance 320 is positioned within the mouth of the user, and controller 352, power source 354, accelerometer 356, and transceiver 362 are located generally within external portion 358, as illustrated. Accordingly, controller 352, power source 354, accelerometer 356, and transceiver 362 are generally positioned externally to the user when dental appliance 320 is positioned within the mouth of the user, which may reduce any physical impacts of the controller 352, power source 354, accelerometer 356, and transceiver 362 on the user.

Controller 352 may include a microprocessor, memory, analogue-digital (A/D) converter, clock, input/output (I/O) ports, and so forth, in various implementations. The controller 352 may communicate with the power source 354, accelerometer 356, and transceiver 362 as well as with electronic circuitry 380 including sensor 384 to receive data from the power source 354, accelerometer 356, transceiver 362, or electronic circuitry 380 including sensor 384. The data, for example, may be indicative of the status of the power source 354, accelerometer 356, transceiver 362, or electronic circuitry 380 including sensor 384, or may be indicative of the operation of the power source 354, accelerometer 356, transceiver 362, or electronic circuitry 380 including sensor 384. The data may include sensor data indicative of a parameter being sensed by the sensor 384 such as body temperature, respiration, heart rate, and so forth.

The controller 352 may communicate with the power source 354, accelerometer 356, and transceiver 362 as well as with electronic circuitry 380 including sensor 384 to control the operation of the power source 354, accelerometer 356, transceiver 362, or electronic circuitry 380 including sensor 384. For example, the controller 352 may regulate the flow of power from power source 354 to accelerometer 356, transceiver 362, or electronic circuitry 380 including sensor 384. The controller 352 may control the collection of data by the accelerometer 356 or sensor 384. The controller 352 may acquire data from the accelerometer 356 or from the sensor 384, the controller 352 may process the data acquired from the accelerometer 356 or sensor 384. The controller 352 may communicate data to the transceiver 362 for transmission. The controller 352 may regulate the transmission of data by transceiver 362 including data from the accelerometer 356 or sensor 384. The controller 352 may receive data communicated from the transceiver where such data is received by the transceiver 362 from some external source such as remote station 390 (see FIG. 6). Data so received may alter the operation of the controller 352, or may alter, for example, the operation of power source 354, accelerometer 356, transceiver 362, or electronic circuitry 380 including sensor 384, as may be directed by controller 352.

The power source 354, in the implementation illustrated in FIG. 6, is adapted to flow electrical power to the controller 352, accelerometer 356, transceiver 362, and electronic circuitry 380 including sensor 384. The flow of power from the power source 354 to the controller 352, accelerometer 356, transceiver 362, or electronic circuitry 380 including sensor 384 may be controlled by the controller 352. In various implementations, the power source 354 may include a battery, and the battery may be, for example, Ni—Cd, Ni-Metal Hydride, or Li-ion based. The battery portion of the power source 354 may be placed within external portion 358 of electronics package 350 so that the battery is positioned externally to the user when the user is wearing mouthguard apparatus 300.

Accelerometer 356 may measure the acceleration data indicative of acceleration of the user in one or more dimensional space. Accelerometer, as used herein, includes devices that measure acceleration, position, or velocity, and the remaining parameters of position, velocity, acceleration may be derived from these measurements. For example, if the accelerometer measures position over time, the remaining parameters of velocity (rate of change of position with respect to time) and acceleration (rate of change of velocity with respect to time) may be derived from the measured position vs. time data. Depending upon the particular usage of accelerometer herein, (i.e. whether the accelerometer is measuring position, velocity, or acceleration) appropriate adjustment of the accelerometer data may then be made to derive the position, velocity, or acceleration of the user therefrom, as would be readily understood by those of ordinary skill in the art upon study of this disclosure. Accelerometer, as used herein, further includes other devices that may detect an impact upon the user. In various examples in this disclosure, the accelerometer may be a sensor that measures acceleration, but this is exemplary only and not limiting.

Because the accelerometer 356 is secured to the upper mandible in this implementation, the accelerometer is fixed to the head of the user, and, thus may measure accelerations experienced by the head, neck, or other portions of the user or may measure the acceleration of the user in toto. Motions of the head are transmitted to accelerometer 356, which is in secured engagement to the head, in this implementation.

For example, accelerometer 356 may measure acceleration data in three-dimensional space with 6 degrees of freedom—i.e. linear acceleration along the x, y, and z axes and rotational acceleration about the x, y, and z axes. Velocity data indicative of the velocity of the user may be determined from the acceleration data as measured by the accelerometer 356, in various implementations, and the controller 352 may determine the velocity data from the acceleration data. In various implementations, position data indicative of the position of the user may be determined from the acceleration data, and the controller 352 may determine the position data from the acceleration data.

In various implementations, the acceleration data, data derived from the acceleration data such as velocity data or position data, or both, may be indicative of stresses upon the user such as those induced by walking, running, or other activity. In various implementations, the acceleration data, data derived from the acceleration data such as velocity data or position data, or both, may be indicative of work produced by the user as the result of user activity. The stress on the user or the work produced by the user may be indicative of the health of the user, or the level of effort expended in, for example, an athletic training regimen.

In various implementations, the acceleration data, data derived from the acceleration data such as velocity data or position data, or both, may be indicative of impact upon the user such as impacts resulting from participation in a contact sport such as football, hockey, lacrosse, and so forth. Because the sensor is generally secured to the upper mandible, the accelerometer data may be indicative of the magnitude of a particular impact to the user, including impacts to the head and neck area of the user, and the resultant displacement of the head, neck, or both head and neck and the rate of displacement of the head, neck, or both the head and neck, for example, linearly along the x, y, and z axes and rotationally about the x, y, and z axes. This, in turn, may be indicative of injury to the user or the potential for injury to the user from the impact. The location of the accelerometer 356 with respect to the user's spinal column, brain cavity, and other sensitive anatomical structures may be generally determined in various ways in order to relate acceleration data measured by accelerometer 356 to corresponding displacements of the head, neck, and so forth of the user and, thus, to indications of injury to the user.

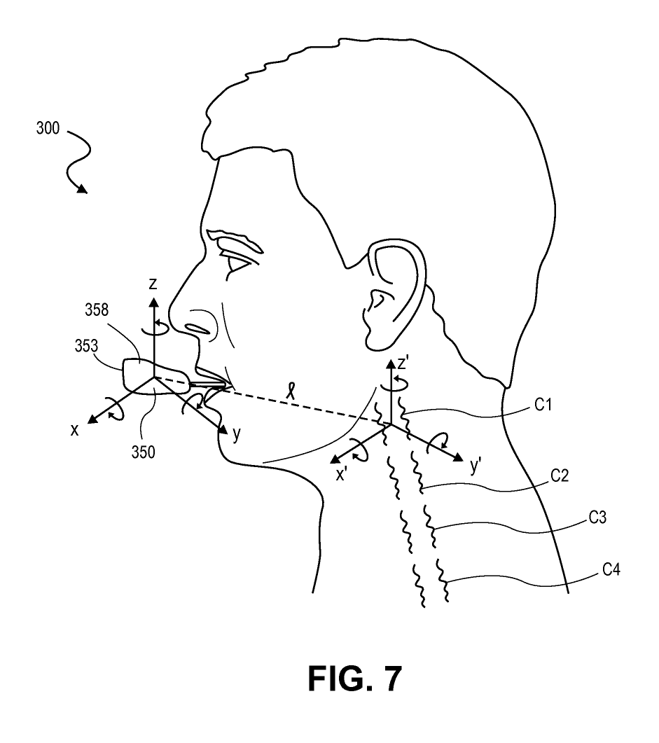

For example, as illustrated in FIG. 7, the dental appliance 320 (not visible in FIG. 7) is positioned within the mouth and secured to the teeth of the user. The electronics package 350 is secured to dental appliance 320 and extends past the lips of the user such that portions of the electronics package 350 including end 353 form an external portion 358 of electronics package 530 that is positioned external to the mouth of the user, as illustrated. Accelerometer 356, which is positioned in external portion 358 of electronics package 350, is generally in mechanical communication with the teeth of the upper jaw to which the dental appliance 320 is secured, so that accelerations measured by accelerometer 356 are indicative of accelerations of the upper jaw, and, thus, of the user. Accelerometer 356 measures linear acceleration along the x, y, and z axes and rotational acceleration about the x, y, and z axes, as illustrated. Accelerometer 356 is generally positioned at distance l from the user's Cl vertebrae. Accordingly, if distance l is generally known, the linear accelerations along the x′, y′, and z′ axes and the rotational accelerations about the x′, y′, and z′ axes centered at the Cl vertebrae may be derived from the linear acceleration along the x, y, and z axes and rotational accelerations about the x, y, and z axes as determined by accelerometer 356. In various implementations, three linear sensors that are not aligned about a single point may be used to measure rotational acceleration.

With reference to FIG. 6, electronic circuitry 380 including sensor 384 may be located about dental appliance 320 and may be in communication with electronic components 360 of electronics package 350. For example, power source 354 may flow power upon electronic circuitry 380 including sensor 384, controller 352 may control the operation of electronic circuitry 380 including the measurement of data by sensor 384, and data measured by sensor 384 may be communicated via electronic circuitry 380 to transceiver 362 for transmission. Electronic circuitry 380 may support various electronic components 360, so that portions of electrical components 360 are apportioned over portions of electronic circuitry 380.

Sensor 384, illustrated in FIG. 6, may detect data indicative of the user status of the user. For example, sensor 384 may detect data indicative of physiologic indicators of user status such as, for example, the user's body temperature, heart rate, cardiac rhythm, breathing rate, CO₂ in the user's breath, or various other physiologic indicators or combinations of physiologic indicators that may be present in the user's breath or saliva or otherwise detectable about the user's mouth or head. The physiologic indicators may indicate that the user is experiencing health problems or is excessively physically stressed due to exercise or activity. For example, the user's body temperature may be indicative of the user's status as being hyperthermic or hypothermic. As another example, heart rate in excess of some maximum heart rate value may be indicative of the user's status as being excessively physically stressed. Various cardiac arrhythmias may be detected by sensor 384, in some implementations. Sensor 384 may communicate data indicative of the status of the user to the transceiver 362.

Transceiver 362 may transmit signals indicative of the operation of the electrical components 360 within electronics package 350, or the operation of the electronic circuitry 380 including sensor 384 within dental appliance 320. In various implementations, the transceiver may transmit signals indicative of acceleration data measured by accelerometer 356 or data derived from the acceleration data such as velocity data or position data to remote station 390. In various implementations, the transceiver 362 may receive signals from the remote station 390 that regulate the operation of the electrical components 360, electronic circuitry 380, sensor 384, or combinations thereof, and the transceiver 362 may distribute such signals received from remote station 390 as appropriate within electronics package 350, electronic circuitry 380, and sensor 384.

Signals may pass between transceiver 362 and remote station 390 via electromagnetic wave. Remote station 390 may be a computer, any of various hand held devices such as a cell phone or iPad®, a dedicated monitoring apparatus, combinations thereof, or so forth. Remote station 390 may be positioned at any suitable location with respect to the user. Remote station 390 may process signals received from the transceiver 362 to derive data therefrom. Remote station 390 may transmit signals to the transceiver 362 to control the operation of the electronics package 350, electronic circuitry 380, or sensor 384, in various implementations. The signals may pass through the electromagnetic spectrum between transceiver 362 and remote station 390, and the signals may conform to various standards such as CDMA2000, Bluetooth™, IEEE 802.11, and so forth, in various implementations.

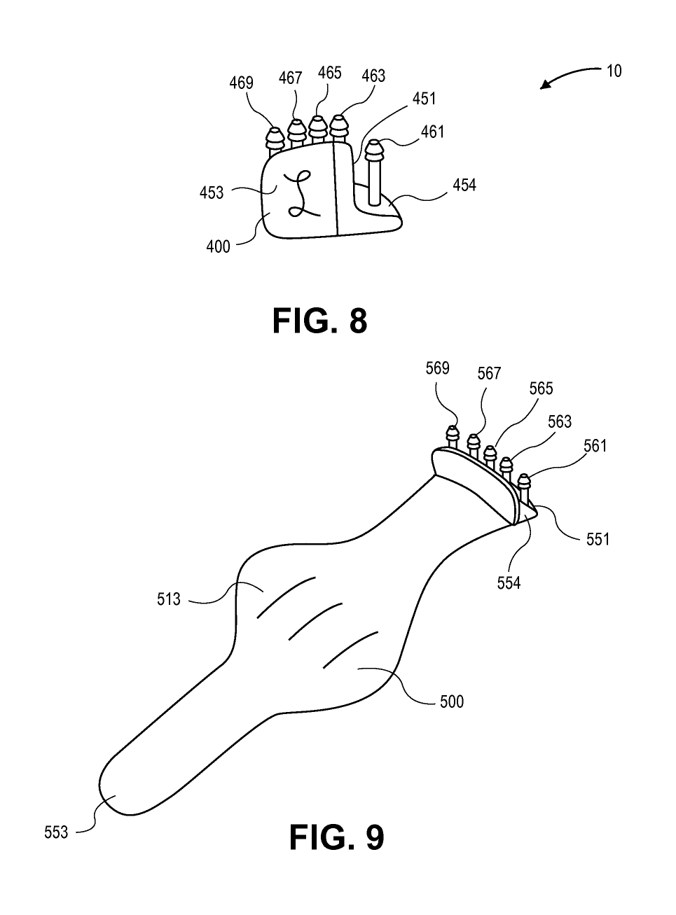

FIG. 8 illustrates plug 400 that may replace electronics package 50 in mouthguard apparatus 10. As illustrated in FIG. 8, plug 400 includes male fittings 461, 463, 465, 467, 469 that may be removably received within female fittings 71, 73, 75, 77, 79, respectively of dental appliance 20 to secure plug 400 to dental appliance 20 with surfaces 451, 454 biases against surfaces 71, 74 of dental appliance 20. Plug 400 is positioned entirely within the mouth when received in dental appliance 20 and dental appliance 20 is positioned within the user's mouth. A display surface 453 may display a team logo, and a number of plugs may be provided that may be interchanged with dental appliance 20, for example, to display various displays upon display surfaces 453 in concert with dental appliance 20. Plug 400 includes no electronics, in this implementation, but may confer mechanical, decorative, or protective attributes to dental apparatus 20 when inserted therein. In other implementations, the plug, such as plug 400, may include an electronics package, such as electronics package 350.

FIG. 9 illustrates handle 500 that may be removably inserted into dental appliance 20 of mouthguard apparatus 10. As illustrated in FIG. 9, handle 500 includes male fittings 561, 563, 565, 567, 569 that may be removably received within female fittings 71, 73, 75, 77, 79, respectively of dental appliance 20 to secure handle 500 to dental appliance 20 with surfaces 551, 554 biases against surfaces 71, 74 of dental appliance 20. Handle 500 may be used to place dental appliance 20 into hot water during the fitment process to soften the occlusal pad material of the occlusal pad into the compliant state. With the occlusal pad material in the compliant state, handle 500 may be used to remove dental appliance 20 from the hot water and then position dental appliance 20 in the mouth for fitment. Wings 513 are provided on handle 500 to assist the user in manipulation of handle 500 with dental appliance 20 attached thereto during the fitment process. Following fitment, handle 500 may be removed from dental appliance 20 and replaced with, for example, electronics package 50 or plug 400.

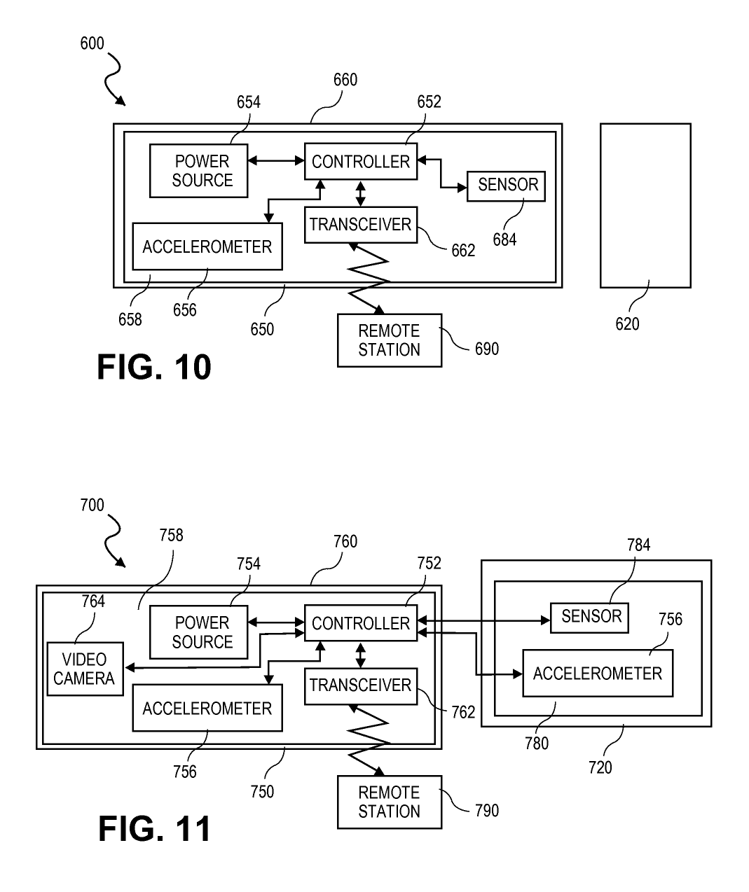

FIG. 10 illustrates an implementation of mouthguard apparatus 600 that includes electronics package 650. The electronic components 660 within electronics package 650 include controller 652, power source 654, accelerometer 656, sensor 684, and transceiver 662 in this illustrative implementation, but may include other combinations of electronic components in other implementations. Dental appliance 620 is shown schematically in FIG. 10, is devoid of electronic circuitry or other electronic components. All electronic components are disposed within electronics package 650 in this implementation illustrated in FIG. 10. Electronics package 650, in this implementation, may be mechanically attached to or detached from dental appliance 620, and dental appliance 620 may be secured to the teeth of the upper jaw of the user to secure electronics package 650 to the upper jaw of the user. In the event of failure of one or more electronic components 660, electronics package 650 may be removed from dental appliance 620 and another electronics package 620 secured to dental appliance 620 without the need for replacement of both the failed electronics package 650 and the still functional dental appliance 620. In the event of failure of dental appliance 620, the electronics package 650 may be removed from dental appliance 620 and secured to another dental appliance 620 without the need for replacement of both the still functional electronics package 650 and the failed dental appliance 620. Accordingly, the electronics package 650 and the dental appliance 620 are fungible. When either the electronics package 650 or the dental appliance 620 wear out or otherwise need replacement, the electronics package 650 may be replaced with a substitute electronics package 650 or the dental appliance 620 may be replaced with a substitute dental appliance 620. The substitute dental appliance 620 connects to the electronics package 650, and the substitute electronics package 650 connects to the dental appliance 620, in various implementations.

In various implementations, components of the electronics package, such as electronics package 650, may be replaceable. For example, if the power source 354 becomes exhausted, at least portions of the power source 354, such as a battery, may be replaced. In other implementations, various sensors such as accelerometer 356 may be replaced with a replacement sensor, and the replacement sensor may sense various user status that may differ from the user status sensed by the sensor.

Electronics package 650 may be formed similarly to electronics packages 50, 250, and dental appliance 620 may be formed similarly to dental appliance 20, in various implementations. Electronics package 650 includes an external portion 658. External portion 658 of electronics package 650 may be generally positioned externally to the mouth when dental appliance 620 is positioned within the mouth of the user, and controller 652, power source 654, accelerometer 656, sensor 684, and transceiver 662 are located generally within external portion 658, as illustrated. Accordingly, controller 652, power source 654, sensor 684, accelerometer 656, and transceiver 662 are generally positioned externally to the user when dental appliance 620 is positioned within the mouth of the user, which may reduce any potential physical impacts of the controller 652, power source 654, accelerometer 656, and transceiver 662 on the user. In various implementations, at least a portion of the electronic components 660 of electronics package 650, such as sensor 684 or accelerometer 656, may be positioned within the mouth of the user when electronics package 650 is secured to dental appliance 620 and dental appliance 620 is secured to the jaw of the user.

Power source 654, accelerometer 656, sensor 684, and transceiver 662 may communicate with one another as generally indicated in FIG. 10 as directed by controller 652. Transceiver 662 may communicate with remote station 690 to communicate sensor data from sensor 684 to remote station 690, to communicate acceleration data from accelerometer 656 to remote station 690, and to receive signals from remote station 690 that may direct the operation of electronics package 650 including controller 652, power source 654, accelerometer 656, sensor 684, and transceiver 662.

Accelerometer 656 is included in electronics package 650, electronics package 650 is secured to dental appliance 620, and dental appliance 620 is secured to the jaw in this implementation. Accordingly, in this implementation, accelerometer 656 is fixed to the head of the user, and, thus may measure accelerations of the head, neck, or other portions of the user. Accelerometer 656 may measure linear acceleration, angular acceleration, combinations thereof, and may measure linear acceleration or angular acceleration or both in one dimension, two dimensions, or three dimensions, in various implementations. Sensor 684, in this implementation, measures the user status of the user, and sensor 684 is included in electronics package 650, in this implementation.

FIG. 11 illustrates an implementation of mouthguard apparatus 700 that includes electronics package 750. The electronic components 760 within electronics package 750 include controller 752, power source 754, video camera 764, and transceiver 762, in this illustrative implementation, but may include other combinations of electronic components in other implementations. Dental appliance 720, illustrated schematically in FIG. 11, includes electronic circuitry 780, which is located on or about the material layer(s) that make up dental appliance 720. Electronic circuitry 780 includes accelerometer 756 and sensor 784 in this implementation.

Electronics package 750, in this implementation, may be mechanically attached to or detached from dental appliance 720, and dental appliance 720 may be secured to the jaw of the user to secure electronics package 750 and electronic circuitry 780 to the jaw of the user. When dental appliance 720 is secured to the jaw of the user, external portion 758 of electronics package 750 may be generally positioned externally to the mouth, and controller 752, power source 754, video camera 764, and transceiver 762 are located generally within external portion 758, as illustrated. Accordingly, controller 752, power source 754, video camera 764, and transceiver 762 are generally positioned externally to the user when dental appliance 720 is secured to the jaw of the user, which may reduce the effects of the controller 752, power source 754, video camera 764, and transceiver 762 on the user. Sensor 784 and accelerometer 756 are positioned within the mouth of the user and secured to the jaw of the user when dental appliance 620 is secured to the jaw of the user in this implementation.

Power source 754, accelerometer 756, sensor 784, video camera 764, and transceiver 762 communicate with one another as generally indicated in FIG. 11 as directed by controller 752. Transceiver 762 may communicate with remote station 790 to communicate sensor data from sensor 784 to remote station 790, to communicate acceleration data from accelerometer 756 to remote station 790, to communicate images from video camera 764, and to receive signals from remote station 790 that may direct the operation of electronics package 750 including controller 752, power source 754, accelerometer 756, sensor 784, video camera 764, and transceiver 762.

Video camera 764, which is positioned in external portion 758 of electronics package 750, may be configured to capture images, sound, or both images and sound, in various implementations. Video camera 764 may include memory for the capture of images for later viewing, may be configured to transmit images in real time to remote station 790, or both, in various implementations. Transceiver 762 may transmit the images to the remote station.

Accelerometer 756 is included in electronic circuitry 780, which is integral with dental appliance 720, and dental appliance 720 is secured to the jaw in this implementation. Accordingly, in this implementation, accelerometer 756 is fixed to the head of the user, and, thus may measure accelerations of the head, neck, or other portions of the user. Sensor 784, in this implementation, measures the user status of the user, and sensor 784 is included in electronic circuitry 780, in this implementation. Sensor 784 may be positioned about electronic circuitry 780 and thus with respect to dental appliance 720 to contact the body of the user at a particular position or otherwise positioned about the electronic circuitry in ways that may enhance the ability of sensor 784 to detect the user status of the user when dental appliance 720 is secured to the jaw of the user.

In the event of failure of one or more electronic components 760, electronics package 750 may be removed from dental appliance 720 and another electronics package 720 secured to dental appliance 720. In the event of failure of dental appliance 720 or of electronic circuitry 780, the electronics package 750 may be removed from dental appliance 720 and secured to another dental appliance 720.

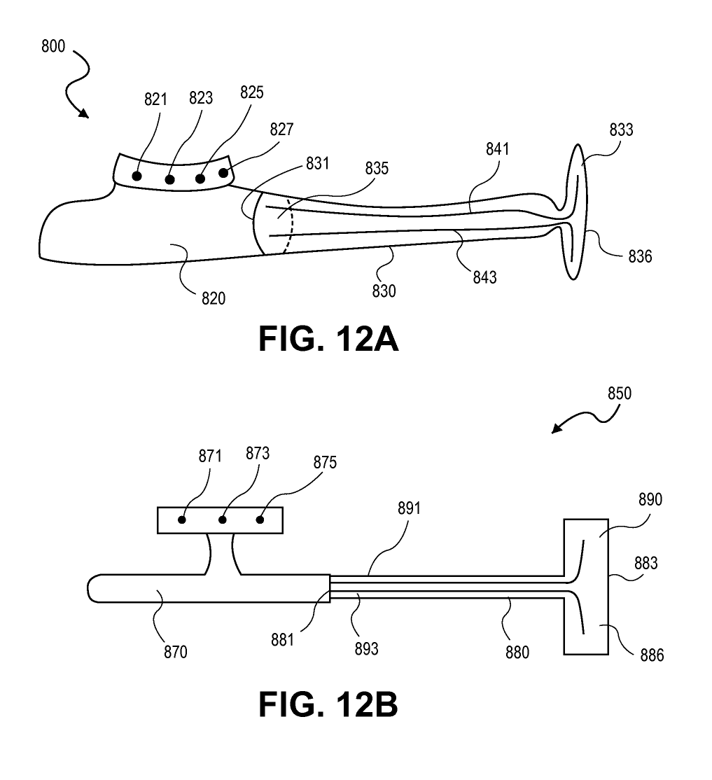

FIG. 12A illustrates an implementation of portions of mouthguard apparatus 800 including electronics package 820. Male connectors 821, 823, 825, 827 allow for attachment of electronics package 820 to a dental appliance (not shown), and the dental appliance may be secured to the user's jaw so that electronics package 820 is secured to the user's jaw accordingly. In various implementations, the electronics package 820 may electrically communicate with electronic circuitry including various sensors integrated with the dental appliance, and may so electrically communicate, at least in part, through male connectors 821, 823, 825, 827. End 831 of strap 830 includes sleeve portion 835 that receives portions of electronics package 820 therein to attach strap 830 thereto. End 831 of strap 830 may be attached to electronics package 820 in various other ways in other implementations. End 833 of strap 830, which is opposite of end 831 of strap 830, is formed as attachment 836 that may be secured to a helmet, including other pieces of equipment or clothing, to secure strap 830 in order to attach the electronics package 820 and the dental appliance, if attached to electronics package 820, to the helmet or other piece of equipment or clothing. Pathways 841, 843, which may be electrically conductive, fiber optic, or suchlike, are located about strap 830 in communication with electronics package 820, as illustrated, to communicate power, signals, or both power and signals between electronics package 820 and end 833 of strap 830. Pathways 841, 843 proximate end 833 of strap 830 may communicate with one or more devices including sensors located about the helmet, clothing, equipment, or the body of the user or otherwise disposed about the user in various implementations to allow the one or more devices to communicate with the electronics package 820 or with electronic circuitry integrated with the dental appliance (if any), or both electronics package 820 and electronic circuitry integrated with the dental appliance through pathways 841, 843 of strap 830.

FIG. 12B illustrates an implementation of portions of mouthguard apparatus 850 including electronics package 870. Male connectors 871, 873, 875 allow for attachment of electronics package 820 to a dental appliance (not shown), and the dental appliance may be secured to the user's jaw so that electronics package 870 is secured to the user's jaw accordingly. In various implementations, the electronics package 870 may electrically communicate with any electronic circuitry including various sensors that may be integrated with the dental appliance, and may so electrically communicate, at least in part, through male connectors 871, 873, 875. End 881 of strap 880 is attached to electronics package 870. End 883 of strap 880, which is opposite of end 881 of strap 880, is formed as attachment 886 that may be secured to a helmet or other piece of equipment or clothing to secure strap 880 thereto. Electronics package 890 is located at end 883 of strap 880, as illustrated. Various electronic components such as a controller, power source, accelerometer, sensor, or transceiver, or combinations thereof, may be apportioned between electronics package 870 and electronics package 890, and the electronic components may be apportioned between electronics package 870 and electronics package 890 in various ways in various implementations. Pathways 891, 893, which may be electrically conductive, fiber optic, etc., are located about strap 880 in communication with electronics package 820, as illustrated, to communicate power, signals, or both power and signals between electronics package 820 and electronics package 890. Fewer or additional pathways, such as pathways 891, 893 or pathways 841, 843, may be provided through the strap, such as strap 830, 880, in various other implementations.

In operation, a mouthguard apparatus, such as mouthguard apparatus 10, 300, 600, 700, 800, 850 may be positioned within the user's mouth and the electronics package, such as electronics package 50, 250, 250, 350 activated. Various switches and so forth may be provided to allow activation of the electronics package by allowing the flow of power therein. The user may then engage in various activities with the mouthguard apparatus positioned in the mouth and the electronics package activated. The mouthguard apparatus may detect the user status of the user including physiologic condition(s) of the user, impact(s) on the user, and so forth. Various sensors, such as sensor 84, 86, 384, 684, 784 and accelerometer 356, 656, 756, may be provided within the dental appliance, within the electronics package, or both within the electronics package and the dental appliance to detect the user status of the user. The mouthguard apparatus may transmit signal(s) indicative of the user status of the user to a remote station, such as remote station 390, 690, 790. The remote station may analyze the signal(s). The remote station may transmit signal(s) to the mouthguard apparatus to control the operation of the mouthguard apparatus including the detection of data and the transmission of data. A coach, physician, or other personnel may use the data from the mouthguard apparatus to monitor for injury, physical distress, level of exertion, and so forth, in various implementations.

Alternate electronics packages may be attached to the dental appliance, such as dental appliance 20, 320, 620, 720. For example, if the dental appliance should fail, the electronics package may be removed from the failed dental appliance and attached to a functional dental appliance. Alternatively, should the electronics package fail, the failed electronics package may be removed from the dental appliance and a function electronics package attached to the dental appliance. In various implementations, the dental appliance, the electronics package, or both the dental appliance and the electronics package may be generally disposable. Various electronics packages, handles, such as handle 500, and plugs, such as plug 400, may be interchanged with the dental appliance as the user desires. Various combinations of electronics packages, handles, plugs, and dental appliances that may be interchangeable with one another in various combinations may be provided, and the functional characteristics of the mouthguard apparatus may be varied by combination.

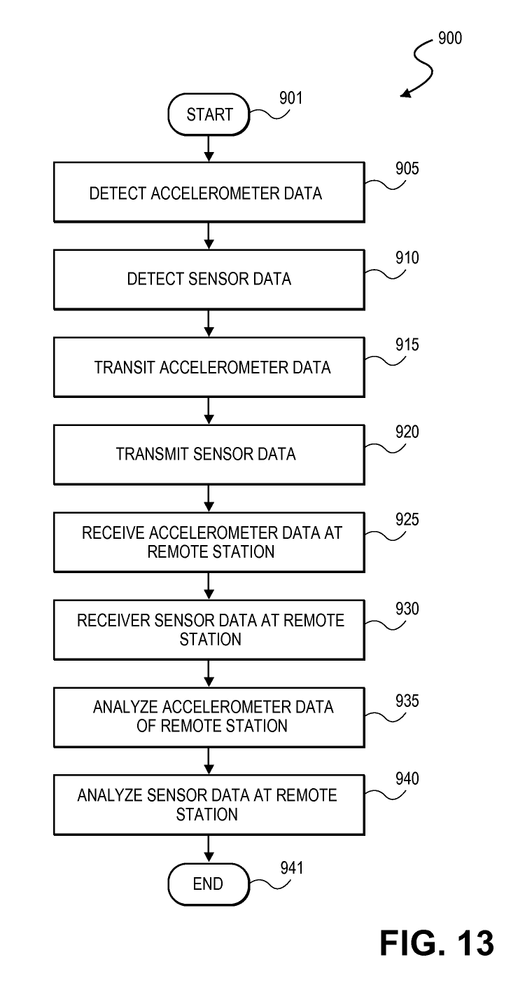

An exemplary method of use of the mouthguard apparatus 900 is illustrated by flow diagram in FIG. 13. As illustrated in FIG. 13, method 900 starts at step 901. At step 905 a sensor configured as an accelerometer detects accelerometer data indicative of acceleration. The data may be indicative of linear acceleration or may be indicative of radial acceleration, or combinations thereof, and the data may have any dimensionality such as, for example, with respect to an x, y, or z coordinate axis, or combinations of the x, y, or z coordinate axes. At step 910, the sensor may detect sensor data indicative of the user status of the user. At step 915 the transceiver, such as transceiver 362, transmits the accelerometer data to the remote station. At step 920 the transceiver transmits the sensor data to the remote station. At step 925 the remote station receives the accelerometer data, and the remote station receives the sensor data at step 930. At step 935 the remote station analyses the accelerometer data. The remote station may determine velocity, position, impact on the user, the user's response to impact, and so forth at step 935. The remote station may cooperate with controller, such as controller 352, in the electronics package in determining, for example, the velocity, position, impact on the user, and the user's response to impact. The remote station analyzes the sensor data to determine user status at step 940. The remote station may cooperate with the controller to determine user status. Method 900 terminates at step 941. The remote station, the controller, or both may direct the collection of data, the transmission of data, and the operation of the mouthguard apparatus in various implementations. The remote station may be positioned generally proximate the user, for example, proximate an athletic field upon which the user is engaged in athletic activity, in some implementations. The remote station may be positioned at a geographically remote local from the user in other implementations.

The foregoing discussion along with the Figures discloses and describes various exemplary implementations. These implementations are not meant to limit the scope of coverage, but, instead, to assist in understanding the context of the language used in this specification and in the claims. Upon study of this disclosure and the exemplary implementations herein, one of ordinary skill in the art may readily recognize that various changes, modifications and variations can be made thereto without departing from the spirit and scope of the inventions as defined in the following claims.

What is claimed is:

1. A mouthguard apparatus, comprising:
an electronics package adapted to attach removably to a dental appliance, the dental appliance configured to be secured within the mouth of a user, at least portions of the electronics package adapted to extend external to a mouth of the user when the electronics package is attached to the dental appliance and the dental appliance is secured within the mouth of the user.

2. The apparatus, as in claim 1, further comprising:
a power source disposed about the electronics package to provide electrical power thereto, the power source is positioned external to the mouth of the user with the dental appliance secured within the mouth of the user.

3. The apparatus, as in claim 1, further comprising:
a transceiver disposed about the electronics package and adapted to communicate with a remote station.

4. The apparatus, as in claim 1, wherein the electronics package is adapted to detect a physiological state of the user.

5. The apparatus, as in claim 1, wherein the electronics package is adapted to detect a position of the user.

6. The apparatus, as in claim 1, wherein the electronics package is adapted to detect an impact.

7. The apparatus, as in claim 7, further comprising:
an electronic circuit disposed about portions of the dental appliance and adapted to communicate electrically with the electronics package.

8. The apparatus, as in claim 8, wherein the electronic circuit cooperates with the electronics package to detect a physiological state of the user.

9. The apparatus, as in claim 8, wherein the electronic circuit cooperates with the electronics package to detect a position of the user.

10. The apparatus, as in claim 8, wherein the electronic circuit cooperates with the electronics package to detect an impact.

11. The apparatus, as in claim 1, with the dental appliance comprising:
an occlusal pad comprised of an occlusal pad material transformable between a compliant state and a non-compliant state, the occlusal pad defines a channel adapted to engage one or more teeth of the user.

12. The apparatus, as in claim 12, wherein the occlusal pad material comprises a material chosen from a group consisting of polycaprolactone, ethylene vinyl acetate, and polyolefin elastomer.

13. The apparatus, as in claim 1, with the dental appliance comprising:
a base comprised of a base material.

14. The apparatus, as in claim 14, wherein the base material comprises a material chosen from the group consisting styrene block copolymer, ethylene vinyl acetate, polyolefin elastomer, thermoplastic rubber, polypropylene, and injection molding resin.

15. A mouthguard apparatus, comprising:
a dental appliance, at least a portion of the dental appliance configured to be positioned within the mouth of a user, the dental appliance defining at least one bite surface, the bite surface configured to be positioned about teeth of the user;
an electronics package attached removably to the dental appliance such that at least portions of the electronics package extend external to the mouth of the user.

16. The apparatus, as in claim 15, further comprising: a transceiver disposed about the electronics package and in electrical communication with the monitoring element to transmit the signal.

17. The apparatus, as in claim 15, wherein the power source is disposed about the electronics package, at least portions of the power source adapted for positioning external to the mouth of the user.

18. The apparatus, as in claim 15, wherein the dental appliance further comprises:
an occlusal pad comprised of an occlusal pad material transformable between a compliant state and a non-compliant state, the occlusal pad defines a channel adapted to engage one or more teeth of the user.

19. The apparatus, as in claim 15, wherein the dental appliance may be replaced by a replacement dental appliance and the electronics package may be replaced with a replacement electronics package.

20. A method, comprising the step of:
detecting accelerometer data indicative of an impact upon a user using a sensor secured to a dental appliance, the dental appliance removably secured to the teeth of the user, the sensor receiving power from a power source removably attached to the dental appliance and positioned external to the mouth of the user.
